(19) United States
(12) Patent Application Publication
(10) Pub. No.: US 2010/0143331 A1
(43) Pub. Date: Jun. 10, 2010

(54) METHOD FOR MIXING POWDERS

(75) Inventors: Torsten Schultz-Fademrecht, Maselheim (DE); Sandra Zimontkowski, Tuebingen (DE); Patrick Garidel, Norderstedt (DE); Hans-Joachim Kern, Mittlebiberach (DE); Klaus-Juergen Steffens, Rheinbach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM PHARMA GMBH & CO. KG, Ingelheim (DE)

(21) Appl. No.: 12/514,104

(22) PCT Filed: Nov. 8, 2007
(86) PCT No.: PCT/EP2007/062040

§ 371 Date: Feb. 2, 2010

(30) Foreign Application Priority Data
Nov. 10, 2006 (DE) 10 2006 053 375.5

(51) Int. Cl. A61K 39/395; A61K 9/14; A61P 9/10

(52) U.S. Cl. 424/130.1; 424/489

(57) ABSTRACT

The invention relates to a method for preparing powder mixtures, one component consisting of spray-dried powder. The invention also relates to a method for coating spray-dried particles with nanoscale particles, a method for mixing spray-dried powder with microscale particles and a method for covering carrier substances with spray-dried particles.

### BACKGROUND TO THE INVENTION

1. Technical Field

The invention relates to a process for preparing powder mixtures, wherein one component consists of spray-dried powder. The invention also relates to a method of coating spray-dried particles with nanoscale particles and a method of coating carriers with spray-dried particles.

2. Background

Spray-drying is a very good method for preparing inhalable powders. In this process, particles with an MMAD of <10 μm can be prepared directly in one step. Alternative powder production methods, such as for example freeze-drying or precipitation, generally require a subsequent grinding step.

An essential criterion for the quality of inhalable powders is the flowability and also the dispersibility of the powders. Particularly small particles, i.e. those with a MMAD<10 μm, have a tendency to form particle clumps, thus seriously impairing the inhalation properties of the powders. The reason for this deterioration in the powder characteristics is the fact that as the particle diameter decreases, the Van-der-Waals forces, for example, but also polar interactions and electrostatic forces increase disproportionately compared with the gravitational force of the particles.

The knowledge of this behaviour is part of the prior art, which means that numerous proposed solutions have already been developed.

For example, micronised particles, i.e. particles less than 10 μm, may be applied to carriers. These carriers, for example lactose monohydrate, glucose or mannitol, have substantially larger particle diameters (50-200 μm) compared with micronised particles, as a result of which the influence of the gravitational force on the particle properties increases.

Further strategies for improving the flow and dispersion properties are the increased surface roughness obtained for example by coating the micronised particles with nanoparticles (G. Huber et al., Powder technology 134 (2003) pages 181-192) or rendering the particles hydrophobic, for example by coating them with magnesium stearate or with hydrophobic amino acids (WO200403848).

In these known processes, an additional step is needed after the production of the micronised particles, in which the corresponding powder is mixed with the second component such as, for example, a carrier, nanoparticles or a film-forming agent.

Whereas the mixing of ground crystalline microparticles is conventional and is therefore known in the art, the mixing of spray-dried powders is a technical challenge. Spray-dried powders, particularly protein-containing powders, are usually amorphous. This means that these powders are hygroscopic and compared with the crystalline variant they have a higher surface energy and often also a higher electrostatic charge. These properties seriously interfere with their miscibility with another particle population. The pharmaceutical effects of an inadequate mixing process include for example a reduced or inadequate homogeneity of the dose of therapeutic active substance when administered to the patient.

One method of applying nanoparticles to spray-dried powders is mechanical coating in a jet mill or in a hybridizer (made by Nara). Another possibility is electrostatically assisted mixing, in which the driving force is an opposite electric charge for the spray-dried particle and nanoparticle (G. Huber et al., Powder technology 134 (2003) pp. 181-192).

When mixing spray-dried material with carrier systems, screens or gravity mixers are normally used.

In conventional mixing processes it is difficult to maintain a process chain that operates at reduced relative humidity levels. This aspect is particularly important with amorphous powders. Amorphous powders have a tendency, especially during storage, to absorb water from the environment. As absorbed water lowers the glass transition temperature of the powder and as a result the glass transition temperature is often below the storage temperature, there is an increased tendency for recrystallisation effects. Moreover, as the result of water vapour absorption in the powder, capillary condensation of the water vapour may occur, thus seriously impairing the flowability of the powder.

Another critical point in conventional two-step processes is that active intervention is required in the process. This means that the powder produced is harvested in order to feed it into the mixing apparatus. This intervention detracts from the efficiency and safety of the pharmaceutical manufacturing process.

Moreover, most processes, such as, for example, mixing in a jet mill or ball mill but particularly in a hybridiser, are associated with temperature effects (hot spots) as a result of the mechanical loading. As with the water uptake, by exceeding the glass transition temperature of the powder there may be local recrystallisation of the amorphous spray-dried powder. Furthermore, as a result of the mechanical stresses during mixing, the spray-dried particles may be destroyed by fragmentation or fusion.

Another problem arises from the destruction of the fine structure of nanoscale particles by mechanical loading and by the duration of activity of the mechanical loading during mixing processes. The nanoparticles act as spacers on a spray-dried particle and by reducing the Van-der-Waals forces improve the flowability of the powders and the aerodynamic characteristics. At a high load intensity, a sealed film consisting of the nanoparticles may form on a particle and thus counteract the positive effects. (M. Eber, 2004, Dissertation for the University of Erlangen, Title: Efficacy and Performance of Nanoscale Flow Regulators)

One objective of the present invention is therefore to improve a mixing process with reduced relative humidity levels. A further objective is to reduce the mechanical load and hence hot spots during the mixing process.

Published Patent Application WO03/037303 describes a process in which particle mixtures are prepared from spray-dried powders in a one step process, by spraying two spray media into a drying tower simultaneously. According to this, an additional particle population is produced through a second atomising nozzle and this is subsequently mixed with the powder containing the active substance in the spray dryer.

The disadvantage of this process is that only a very limited range of sizes of the two particle populations can be achieved using this technology. Thus, it will not be possible to coat spray-dried particles containing active substance with nanoscale particles. Moreover, this process cannot be used to prepare mixtures of a crystalline and hence a thermodynamically stable carrier (with a preferred size of between 50-200 μm) and spray-dried powders.

A further objective of the present invention is therefore to mix the spray-dried particles with nanoparticles or with crystalline carriers.

In the spray drying of thermally unstable active substances, particularly proteins, and during the production of powders with a low glass transition temperature, additional problems arise as the protein may be damaged by an unfavourable temperature profile during the spray drying.

Under these conditions, in conventional spray drying the exit temperature has to be reduced, which means that the entire drying process has to be carried out in a suboptimal manner.

One possible method of optimising the drying process is to cool the cyclone. However, a disadvantage of this method is that powder deposits in the cyclone have an insulating effect and may therefore make the cooling slow and inefficient.

A further objective of the present invention therefore relates to the necessary and rapid or efficient limiting of the exit temperature during spray drying while maintaining high entry temperatures.

Producing powders with a high protein charge, e.g. more than 50% (w/w), is particularly problematic. High protein contents have a deleterious effect on the powder properties such as the flowability, for example. The powders generally exhibit a very high tendency to cohesion and adhesion. This means that the yields of the drying process and the subsequent processing steps are affected. Furthermore, powders containing large amounts of therapeutic proteins have poor inhalability owing to the cohesive nature of the proteins. This gives rise to the problem of producing spray-dried powders with high proportions of therapeutic proteins without any negative impact on the powder and inhalation properties.

A completely different approach to optimising the physicochemical properties of spray-dried powders is to alter the powder composition. Thus, by adding hydrophobic substances to the spray solution, the skilled man can modify the particle surface of the resulting particles in order to obtain better dispersing qualities. A drawback of this, however, is that it may lead to incompatibility of the hydrophobic excipients with the active substance. Thus, it is known, for example, that hydrophobic excipients have a higher affinity for denatured than for native proteins. Therefore, protein aggregation may be caused by the interaction of hydrophobic excipients and proteins.

A further disadvantage is that hydrophobic substances in the spray-dried particle often result in undesirable crystallisation effects, which again can cause protein damage. This gives rise to the additional problem that satisfactory flowability or satisfactory dispersion characteristics of spray-dried powders have to be ensured without damage to the protein caused by the effects of crystallisation of the powders.

The aim is therefore to provide a process for producing powder mixtures consisting of spray-dried powders and carriers, respectively, with nanoscale particles, which
1. continuously allows reduced relative humidity,
2. represents a reduction in the mechanical load,
3. is particularly gentle on the particles and favourable to the stability of the active substance and at the same time
4. ensures adequate flowability or adequate dispersion characteristics of the spray-dried powders.

### SUMMARY OF THE INVENTION

The present invention solves the problem by means of a method for mixing nanoparticles, microscale particles or carrier systems which is carried out directly in the spray dryer in a one step process. This is a very gentle process for the spray-dried powder. As the mixing takes place directly in the spray dryer, the mixing process can be carried out at very low relative humidities. There is no need to transfer the powder into a mixing apparatus after spray drying. There is no need for any additional input of energy to disperse the spray-dried powder, as the powder produced is present in optimally dispersed form during the spray drying as a result of the prior atomising of the spray solution.

As a result of the gentle dispersion of the nanoparticles, moreover, there is hardly any destruction of the desired nanostructures.

Additionally, by blowing cool air in after the end of the drying the temperature load on the spray-dried powder can be reduced. As a result the powder is less stressed, which is advantageous particularly with longer process times.

When developing spray-dried powders and particularly powder formulations that contain proteins, the skilled man generally has the problem of achieving both good stability of the active substance and also good powder properties (such as for example good flowability and dispersibility). Particularly when there are high protein contents in the powder, as is necessary for example with high-dose medicaments, the powders have a tendency to very strongly cohesive characteristics. Typical examples of this type of active substance are IgG type antibodies.

As a result of the present invention it is now possible to largely undo the dependency between protein stabilisation and powder properties. The formulation of the spray solution and hence of the powder can be focused essentially on the protein stability. The optimising of the aerodynamic properties, on the other hand, can be achieved by mixing with suitable excipients directly in the spray dryer.

Moreover, by adding an inert excipient, the protein content in the powder mixture and hence the dosage of active substance can be regulated. The preparation of different dosages is made substantially easier by mixing with a carrier.

This also results in reduced process times and lower costs for producing suitable machinery.

By the option of cooling the powder by blowing cool air in after drying the spray solution the powder can be further stabilised so that even thermally unstable substances can be processed more satisfactorily.

One application of the invention is the development of powders, e.g. powder-containing preparations of medicaments, e.g. for inhalation and for nasal or oral applications. Another method of using the powders developed is to dissolve (reconstitute) them in a suitable liquid and subsequently administer them by intravenous, subcutaneous, intramuscular or intraarticular route.

A particularly advantageous embodiment of the process according to the invention is the adjustment of the dosage of the powder mixture using microscale mixing components. The particle size should be less than 10 μm or less than 5 μm, respectively. Ideally, the particle size of the mixing component should be of the same order of magnitude as the spray-dried powder.

Another particularly advantageous embodiment of the process according to the invention is the adjustment of the dosage of the powder mixture using carriers. When producing powder for administration by inhaling, the carrier should have a high proportion (more than 30%) of particles with a particle size of less than 100 μm (cf. Example 7, Table 13).

The addition of nanoscale particles to the spray dryer constitutes a particular challenge, as conventional metering devices such as metering screws, metering strips, metering brushes (manufactured by Palas), vibration channels etc. are unsuitable for nanoscale particles. Therefore, another particularly advantageous embodiment of the process according to the invention consists in the mixing of nanoscale particles into the spray dryer. The adjustment of the mass flow into the spray dryer is carried out pneumatically according to the invention. In this embodiment, the layer of powder of the mixing component in a storage vessel is homogenised by mechanical stirring. The nanoscale particles are converted into the aerosol by a current of air and then fed into the spray dryer through a venturi nozzle. The mass flow is adjusted both by the input of energy during the mechanical stirring and by the volume flow in the storage vessel.

Another particularly advantageous embodiment of the process according to the invention is the mixing of the spray-dried powder with hydrophilic or hydrophobic nanoscale particles.

Examples of suitable nanoscale particles are highly dispersed hydrophilic silicon dioxide or the hydrophobised Aerosil R972. The use of the nanoscale particles is not limited to the silicon dioxides mentioned. The determining factor for the usability of the excipients is the particle size, which should be substantially below 1000 nm, or below 500 nm, and particularly advantageously below 100 nm.

The invention cannot be inferred from the prior art.

In the published Patent Application WO200403848, powders (including spray-dried powders) are mixed with an amino acid, with Mg-stearate and with a phospholipid in a mill (jet mill/ball mill) after manufacture. The purpose of this procedure was to optimise the aerodynamic properties of powders by modifying the particle surfaces (rendering them hydrophobic). The aim of this process is to form films on the particle surface and not to increase the surface roughness. However, increasing the surface roughness is an important aspect of the present invention. Furthermore, using the process described in the patent application it is not possible to combine the manufacture of the spray-dried powders with the surface modification in a one step process.

Published Patent Applications WO2000053158 and WO2000033811 describe mixing processes in which a powder which has already been prepared is mixed with an additional powder by a further mixing process. The aim of these mixing processes is to optimise the powder properties and to apply the powder to a carrier. Screens and gravity mixers (e.g. Turbular mixers) are used as the mixers. Moreover, mixing principles are mentioned in which the mixing is induced by shear stress. However, the processes mentioned in these applications are in every case two-step processes. In other words, in contrast to the present invention, the mixing takes place after the manufacture of the powder in a separate process step.

In another published Patent Application (US2004/0118007, “Methods and apparatus for making particles using spray dryer and in-line jet milling”) a process is described in which the spray-dried powder is fed into a jet mill in-line immediately after being produced. The aim of this additional step is to destroy clumps but not to modify the surface of the spray-dried particles by the addition of carrier systems and nanoparticles. Moreover, with this process it is not possible to create dosages by diluting with carriers.

Published Patent Application WO03/037303 also describes a process in which a powder mixture is produced directly in the spray dryer. In this process, two spray solutions are fed into the drying tower independently of one another through a multiple nozzle. During manufacture, both raffinose and leucine particles are produced. The particles are then mixed in the spray dryer. However, this method cannot be used to prepare mixtures with crystalline carrier systems and nanoparticles.

The document GB866038 describes a method of producing polyvinylacetate powders by spray drying. In this process an inert powder such as calcium carbonate or titanium dioxide is introduced, also in admixture with a so-called plasticiser, during the drying process. The essential point of this process is that the drying process of the polyvinylacetate is only stopped after the inert material has been fed in. The objective is to encapsulate the inert material in the spray-dried particle but not to modify the surface nature by introducing the inert particle. Moreover, this process is not a process of mixing two particle populations but the preparation of new hybrid particles consisting of polyvinylacetate and the inert material.

U.S. Pat. No. 3,842,888 describes a method of preparing borax pentahydrate. In this process, drying is carried out in countercurrent by first pre-drying a detergent solution in the drying tower and then feeding the borax solution which is to be dried into the drying tower underneath the supply of detergent solution. The objective is to produce powder, essentially borax, of low density. In this process no mixing of two different powders takes place. Moreover, this patent does not comprise any feeding of a powder into the drying tower but rather the feeding in of two liquids.

The document JP8302399 describes a process in which detergents are dried by the countercurrent method. In order to improve the yield of the dried detergent granules, an inorganic excipient such as an aluminium silicate is fed into the drying tower. This process differs from the present invention in that by this method detergents are dried by the countercurrent process and not by a cocurrent process. Particularly thermally unstable active substances are damaged during countercurrent drying as a result of the greater exposure to high temperatures. With proteins, denaturing may occur, for example. In addition, the document describes only the spray drying of detergents. The drying of pharmaceutical active substances is not addressed in the document. The conditions for detergents cannot be applied to proteins or pharmaceutical active substances, particularly antibodies, as these are more thermally unstable and delicate.

### DESCRIPTION OF THE FIGURES

All the percentages mentioned in the description relate to concentrations and compositions of the dry solids, particularly in a powder obtained by spray drying (w/w).

FIG. 1: SKETCH OF MODIFIED SPRAY DRYING PROCESS WITH INTEGRAL MIXING UNIT

FIG. 1 shows a diagram of a modified spray drying process. A spray solution containing one or more active substances and one or more excipients is supplied through a pumping device to the

atomiser unit (1). This may be any desired type of atomising system, e.g. twin or triple nozzles, pressurised nozzles, centrifugal nozzles, venturi nozzles or ultrasonic nozzles. After atomisation the droplet formed is evaporated down by a drying gas in the

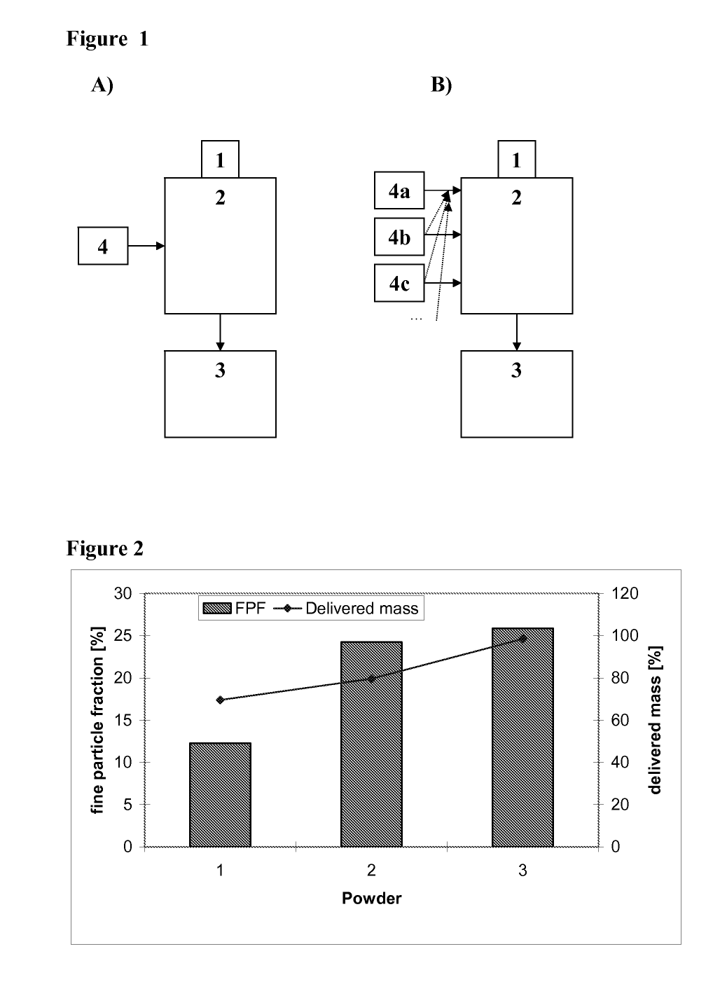

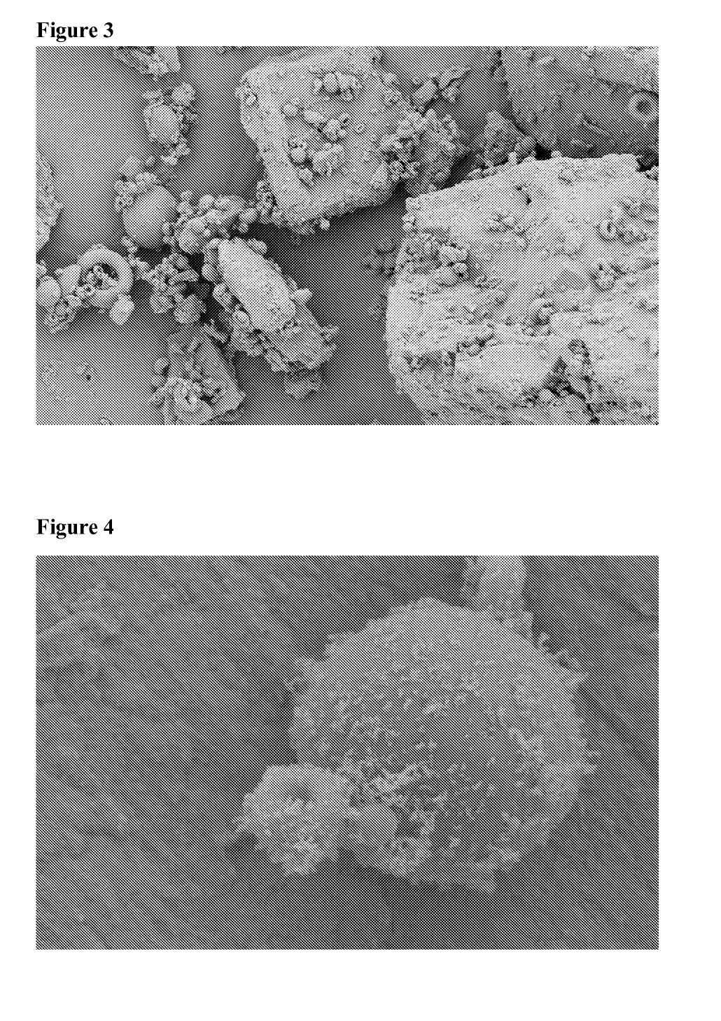

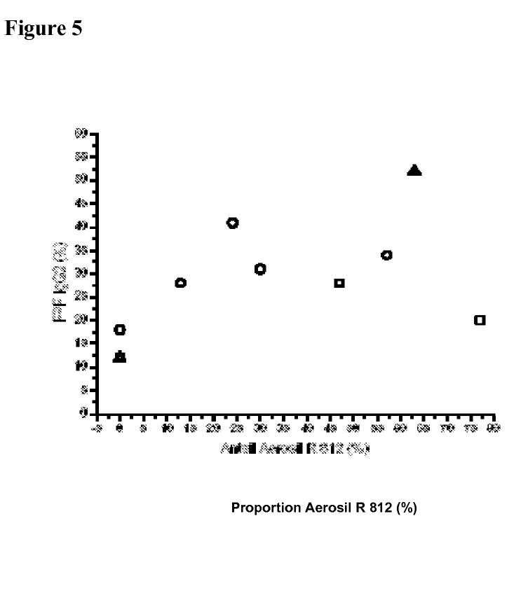

drying tower (2) by the cocurrent process until finally a particle is formed. After the drying process, one (see FIG. 1A) or more (see FIG. 1B) other powders are introduced into the drying tower (2) through one (see FIG. 1A) or more (see FIG. 1B) suitable

dispersing and metering units (4). In the drying tower (2) and in the subsequent

particle collector or gas/solid separator (3), e.g. a cyclone, the two different particles are combined and form a mixture. The mixture may consist, for example, of the spray-dried powder and a powder of nanoscale particles or of spray-dried powder and a carrier, e.g. crystalline lactose. Combinations of spray-dried powders, nanoscale particles and carriers are also encompassed by the invention. Another preferred embodiment is the mixing of different spray-dried powders.

A) Supply of one other powder/carrier/nanoparticle

B) Supply of a number of other powders, carriers or nanoparticles referred to as 4a, 4b and 4c. Dotted arrows indicate an alternative method in which these additional powders, carrier or nanoparticles are first premixed and then fed through a single suitable dispersing and metering unit.

The drying of the drop and the production of the spray-dried powder correspond to the current state of the art. This means that the spray solution may both be aqueous and consist of any desired pharmaceutically acceptable organic solvent. The drying medium is either air or nitrogen. The entry temperatures of the drying gas into the drying tower are between 50° C. and 200° C. The exit temperatures after passing through the drying tower are between 25° C. and 150° C.

FIG. 2: FINE PARTICLE FRACTION (FPF) AND DELIVERED MASS OF DIFFERENT POWDERS (WITH/WITHOUT GRANULAC 140)

The fine particle fraction was determined using a one-step impactor (Impactor Inlet, TSI) combined with an aerodynamic particle sizer (APS, TSI). The separation threshold for the impactor nozzle was 5.0 μm.

For measurement, the powder was packed into size 3 capsules and expelled using an inhaler (HandiHaler®, Boehringer Ingelheim. The flow rate for delivering the powder was adjusted so that a pressure drop of 4 kPa prevailed through the HandiHaler. The air volume was 4 litres according to Pharma Eur. To prevent the particles deposited on the impactor stage from “rebouncing”, the impactor plate was coated with a highly viscous Brij solution during the measurements.

The delivered mass is calculated from the difference in the weight of the capsule before and after the expulsion of the powder from the inhaler.

The fine particle fraction was determined by wet chemistry. For this, the filter on which the fine particle fraction had been deposited was incubated for 3 minutes in a reconstitution medium with gentle tilting. Then the reconstitution medium was filtered sterile and the protein concentration in the filtrate was determined by UV spectroscopy. The results obtained come from three separate measurements.

The bars 1, 2 and 3 represent the three powder mixtures prepared. The mixing ratio of Granulac 140 was 0% (w/w) for powder 1, 30% (w/w) for powder 2 and 90% for powder 3.

FIG. 3: SEM IMAGE—MIXTURE OF SPRAY DRIED POWDER AND A CARRIER

SEM image of spray-dried powder containing a 70% (w/v) IgG1 antibody and 30% (w/v) trehalose and the carrier material Granulac 140 (crystalline lactose monohydrate). Composition of the mixture: 30% spray-dried powder/70% Granulac 140.

The images were taken using a scanning electron microscope (SUPRA 55 VP, made by Zeiss SMT, Oberkochen). The powder samples were sprayed directly onto suitable slides. Excess material was tapped off and blown away. Then the samples were coated with 15 nm gold/palladium to ensure adequate electrical conductivity.

The detection for displaying the images was carried out using secondary electrons.

Magnification: 1000×

Distance of powder from cathode: 10 mm

Shutter size: 10 μm

Accelerating voltage: 5 kV

Vacuum: 4.17e-004 Pa

FIG. 4 SEM IMAGE—MIXTURE OF SPRAY DRIED POWDER AND NANOPARTICLES

SEM image of spray-dried powder containing a 70% (w/v) IgG2 antibody and 30% (w/v) trehalose and the Aerosil R812. Aerosil R812 consists of nanoscale hydrophobic silicon dioxide.

Composition of the mixture: 66% spray-dried powder/24% Aerosil 812R

The images were taken using a scanning electron microscope (SUPRA 55 VP, made by Zeiss SMT, Oberkochen). To do this, the powder samples were sprayed directly onto suitable slides. Excess material was tapped off and blown away. Then the samples were coated with about 15 nm gold/palladium to ensure an adequate electrical conductivity.

The detection for displaying the images was carried out using secondary electrons.

Magnification: 15000×

Distance of powder from cathode: 5 mm

Shutter size: 10 μm

Acceleration voltage: 5 kV

Vacuum: 4.24e-004 Pa

FIG. 5: FINE PARTICLE FRACTION (FPF) DEPENDING ON THE RATIO OF MIX OF NANOPARTICLES AND DISPERSING PRESSURE

This diagram shows different powder mixtures consisting of spray-dried powder (70% (w/v) IgG2/30%(w/v) trehalose) and Aerosil R812. The powders were dispersed at different pressures and introduced into the spray dryer.

Spray drying was carried out using a Büchi B191. The following spray drying conditions were selected:

| | |
|---|---|
| Entry temperature: | 150° C. |
| Exit temperature: | 90° C. |
| Atomiser gas rate: | 700 L/h |
| Aspirator power: | 100% |
| Spray rate: | 3 ml/min |
| Solids content of the spray solution: | 3% |
| Square: | Dispersion pressure 0.5 bar |
| Circle: | Dispersion pressure 1.75 bar |
| Triangle: | Dispersion pressure 3.0 bar |

### DETAILED DESCRIPTION OF THE INVENTION

Terms and designations used within the scope of this specification have the following meanings defined below. The details of weight and percentages by weight are based on the dry mass of the compositions or the solids content of the solutions/suspensions, unless stated otherwise. The general expressions “containing” or “contains” include the more specific term of “consisting of”. Moreover, “one” and “many” are not used restrictively.

“Powder” denotes a very fine, comminuted substance. “Spray-dried powder” means a powder produced by spray drying.

“Particle” denotes a small fragment of a substance. In the present invention the term particles refers to the particles in the powders according to the invention. The terms particles and powders are occasionally used interchangeably in the present invention. The term powder also includes its constituents, the particles. Particles thus refer to all the particles, i.e. the powder.

The term “composition” refers to liquid, semi-solid or solid mixtures of at least two starting materials.

The term “pharmaceutical composition” refers to a composition for administering to the patient.

The term “pharmaceutically acceptable excipients” relates to excipients which may possibly be present in the formulation within the scope of the invention. The excipients may for example be administered by pulmonary route without having any significant toxicologically harmful effects on the subjects or on the subjects' lungs.

The term “mixing” means a process in which different powders are combined in as uniformly distributed a manner as possible. In this process, the particles to be mixed may have the same or different average particle sizes. For example, the term mixing encompasses the combining of two spray-dried powders. The term mixing also encompasses the combining of spray-dried powders with nanoscale particles or with carriers. The term mixing thus also includes the coating of one particle population with a second particle population.

The term “plasticiser” describes a material property according to which this substance lowers the glass transition temperature of an amorphous powder. Thus, for example, water is a plasticiser for spray-dried powders and lowers the glass transition temperature according to the moisture content of the powder.

A one-step process differs from a two-step process in that two process steps are carried out in one unit, e.g. in an apparatus, in a chamber or the like. In a two-step process, 2 units are needed for 2 process steps.

If for example a process consists of a drying step and a mixing step, in a one-step process both the drying and the mixing would take place in one apparatus or in one unit. This unit might be a spray dryer, for example. In a two-step process the powder is transferred after drying from the first apparatus into a second apparatus for the subsequent mixing process. The term “pharmaceutically acceptable salts” includes for example the following salts, but is not restricted thereto: salts of inorganic acids such as chloride, sulphate, phosphate, diphosphate, bromide and nitrate salts. Also, salts of organic acids, such as malate, maleate, fumarate, tartrate, succinate, ethylsuccinate, citrate, acetate, lactate, methanesulphonate, benzoate, ascorbate, para-toluenesulphonate, palmoate, salicylate and stearate, and also estolate, gluceptate and lactobianate salts.

By the term “active substances” are meant substances that provoke an activity or a reaction in an organism. If an active substance is administered to a human or to an animal body for therapeutic purposes, it is referred to as a pharmaceutical composition or medicament.

Examples of active substances are insulin, insulin-like growth factor, human growth hormone (hGH) and other growth factors, tissue plasminogen activator (tPA), erythropoietin (EPO), cytokines, e.g. interleukins (IL) such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18 interferon (IFN)-alpha, -beta, -gamma, -omega or -tau, tumour necrosis factor (TNF) such as TNF-alpha, beta or gamma, TRAIL, G-CSF, GM-CSF, M-CSF, MCP-1 and VEGF. Other examples are monoclonal, polyclonal, multispecific and single chain antibodies and fragments thereof such as for example Fab, Fab′, F(ab′)₂, Fc and Fc′ fragments, light (L) and heavy (H) immunoglobulin chains and the constant, variable or hypervariable regions thereof as well as Fv and Fd fragments (Chamov et al., 1999). The antibodies may be of human or non-human origin. Humanised and chimeric antibodies are also possible. Similarly, it relates to conjugated proteins and antibodies which are connected for example to a radioactive substance or a chemically defined medicament.

Fab fragments (fragment antigen binding=Fab) consist of the variable regions of both chains which are held together by the adjacent constant regions. They may be produced for example from conventional antibodies by treating with a protease such as papain or by DNA cloning. Other antibody fragments are F(ab′)₂ fragments which can be produced by proteolytic digestion with pepsin.

By gene cloning it is also possible to prepare shortened antibody fragments which consist only of the variable regions of the heavy (VH) and light chain (VL). These are known as Fv fragments (fragment variable=fragment of the variable part). As covalent binding via the cystein groups of the constant chains is not possible in these Fv fragments, they are often stabilised by some other method. For this purpose the variable region of the heavy and light chains are often joined together by means of a short peptide fragment of about 10 to 30 amino acids, preferably 15 amino acids. This produces a single polypeptide chain in which VH and VL are joined together by a peptide linker. Such antibody fragments are also referred to as single chain Fv fragments (scFv). Examples of scFv antibodies are known and described, cf. for example Huston et al., 1988.

In past years various strategies have been developed for producing multimeric scFv derivatives. The intention is to produce recombinant antibodies with improved pharmacokinetic properties and increased binding avidity. In order to achieve the multimerisation of the scFv fragments they are produced as fusion proteins with multimerisation domains. The multimerisation domains may be, for example, the CH3 region of an IgG or helix structures (“coiled coil structures”) such as the Leucine Zipper domains. In other strategies the interactions between the VH and VL regions of the scFv fragment are used for multimerisation (e.g. dia-, tri- and pentabodies).

The term “diabody” is used in the art to denote a bivalent homodimeric scFv derivative. Shortening the peptide linker in the scFv molecule to 5 to 10 amino acids results in the formation of homodimers by superimposing VH/VL chains. The diabodies may additionally be stabilised by inserted disulphide bridges. Examples of diabodies can be found in the literature, e.g. in Perisic et al., 1994.

The term “minibody” is used in the art to denote a bivalent homodimeric scFv derivative. It consists of a fusion protein which contains the CH3 region of an immunoglobulin, preferably IgG, most preferably IgG1, as dimerisation region. This connects the scFv fragments by means of a hinge region, also of IgG, and a linker region. Examples of such minibodies are described by Hu et al., 1996.

The term “triabody” is used in the art to denote a trivalent homotrimeric scFv derivative (Kortt et al., 1997). The direct fusion of VH-VL without the use of a linker sequence leads to the formation of trimers.

The fragments known in the art as mini antibodies which have a bi-, tri- or tetravalent structure are also derivatives of scFv fragments. The multimerisation is achieved by means of di-, tri- or tetrameric coiled coil structures (Pack et al., 1993 and 1995; Lovejoy et al., 1993).

The term “excipients” refers to substances which are added to a formulation, in the present invention a powder, particularly a spray-dried powder. Excipients usually have no activity themselves, particularly no pharmaceutical activity, and serve to improve the formulation of the actual ingredient, e.g. an active substance, or to optimise a particular aspect thereof (e.g. storage stability).

A pharmaceutical “excipient” is a part of a medicament or a pharmaceutical composition, and ensures among other things that the active substance reaches the activity site and is released there. Excipients have three basic tasks: a carrier function, controlling the release of active substance and increasing the stability. Excipients are also used to produce pharmaceutical forms which are thereby altered in their duration or rate of effect.

The term “amino acid” refers to compounds which contain at least one amino and at least one carboxyl group. Although the amino group is usually in the a-position to the carboxyl group, any other arrangement in the molecule is conceivable. The amino acid may also contain other functional groups, such as e g amino, carboxamide, carboxyl, imidazole, thio groups and other groups Amino acids of natural or synthetic origin, racemic or optically active (D- or L-) including various stereoisomeric proportions, may be used. For example the term isoleucine includes both D- isoleucine, L-isoleucine, racemic isoleucine and various ratios of the two enantiomers.

The term “peptide”, “polypeptide” or “protein” refers to polymers of amino acids consisting of more than two amino acid groups.

Furthermore the term “peptide”, “polypeptide” or “protein” refers to polymers of amino acids consisting of more than 10 amino acid groups.

The term peptide, polypeptide or protein is used as a pseudonym and includes both homo- and heteropeptides, i.e. polymers of amino acids consisting of identical or different amino acid groups. A “di-peptide” is thus made up of two peptidically linked amino acids, a “tri-peptide” is made up of three peptidically linked amino acids.

The term “protein” used here refers to polymers of amino acids with more than 20 and particularly more than 100 amino acid groups.

The term “small protein” refers to proteins under 50 kD or under 30 kD or between 5-50 kD. The term “small protein” further relates to polymers of amino acid groups with less than 500 amino acid groups or less than 300 amino acid groups or polymers with 50-500 amino acid groups. Preferred small proteins are e.g. growth factors such as “human growth hormone/factor”, insulin, calcitonin or the like.

The term “protein stability” denotes monomer contents of more than 90%, preferably more than 95%.

The term “oligosaccharide” or “polysaccharide” refers to polysaccharides consisting of at least three monomeric sugar molecules.

The term “% (w/w)” refers to the percentage amount, based on the mass, of an active substance or an excipient in the spray-dried powder. The proportion stated is based on the dry substance of the powder. The residual moisture in the powder is thus not taken into consideration.

The term “amorphous” means that the powdered formulation contains less than 10% crystalline fractions, preferably less than 7%, more preferably less than 5%, and most preferably less than 4, 3, 2, or 1%.

The word “inhalable” means that the powders are suitable for pulmonary administration. Inhalable powders can be dispersed and inhaled by means of an inhaler so that the particles enter the lungs and are able to develop a systemic activity optionally through the alveoli. Inhalable particles may have an average particle diameter, for example, of between 0.4-30 μm (MMD=mass median diameter), usually between 0.5-20 μm, preferably between 1-10 μm and/or an average aerodynamic particle diameter (MMAD=mass median aerodynamic diameter) of between 0.5-10 μm, preferably between 0.5-7.5 μm, more preferably between 0.5-5.5 μm, even more preferably from 1-5 μm and most preferably between 1-4.5 μm or 3-10 μm.

“Mass Median Diameter” or “MMD” is a measurement of the average particle size distribution. The results are expressed as diameters of the total volume distribution at 50% total throughflow. The MMD values can be determined for example by laser diffractometry, although of course any other conventional method may be used (e.g. electron microscopy, centrifugal sedimentation).

The term “mean aerodynamic particle diameter” (=mass median aerodynamic diameter (MMAD)) indicates the aerodynamic particle size at which 50% of the particles of the powder normally have a smaller aerodynamic diameter. In cases of doubt the reference method for determining the MMAD is the method specified in this patent specification (cf. The Chapter EXAMPLES, Method).

MMD and MMAD may differ from one another, e.g. a hollow sphere produced by spray drying may have a greater MMD than its MMAD.

The term “fine particle fraction” (FPF) describes the inhalable part of a powder consisting of particles with a particle size of ≦5 μm MMAD. In powders that are readily dispersible the FPF is more than 20%, preferably more than 30%, more particularly more than 40%, and more preferably more than 50%, even more preferably more than 55%. The expression “Cut Off Diameter” used in this context indicates which particles are taken into account when determining the FPF. An FPF of 30% with a Cut Off Diameter of 5 μm (FPF ₅) means that at least 30% of all the particles in the powder have a mean aerodynamic particle diameter of less than 5 μm.

The term “time of flight” is the name of a standard method of measurement, as described in more detail in the Chapter EXAMPLES. In a time of flight measurement the MMAD is determined by measuring the time of flight of a particle over a defined measured distance. The MMAD correlates with the time of flight. This means that particles with a greater MMAD take a longer time to fly than correspondingly smaller particles (cf. on this subject: Chapter EXAMPLES, Method).

The term “dispersible” means capable of flight. The basic prerequisite for the ability of a powder to fly is the disaggregation of the powder into individual particles and the distribution of the individual particles in air. Particle clumps are too big to enter the lungs and are therefore not suitable for inhalation therapy.

The term “delivered mass” states the amount of powder delivered when an inhaler is used. The delivery is determined in this case for example using a capsule, by weighing the capsule before and after the expulsion. The expelled mass corresponds to the difference in mass of the capsule before and after the expulsion.

The term “carrier” means large particles, compared with the spray-dried powder. This property enables the spray-dried powders to be applied to the carrier. If, for example, spray-dried particles are produced having a mean diameter of about 5 μm, the carrier should have a mean particle size of 50-200 μm. Typical carriers are sugars and polyols. The choice of carriers is not, however, limited to these categories of substance.

The term “microscale particles” or “microscale excipients” denotes particles of the same order of magnitude as the particles in the spray-dried powder. The microscale particles are preferably particles with a median aerodynamic particle size (MMAD) of between 0.5-10 μm, 1-10 μm, particularly preferably 2-7.5 μm. Microscale excipients are particularly suitable for preparing powder mixtures in the spray dryer, as the powder components behave aerodynamically similarly and hence unmixing processes are suppressed. Therefore, microscale excipients are preferably suitable for preparing dilutions and doses, particularly with high proportions of spray-dried powder, particularly with high proportions of spray-dried, protein-containing powder.

The term “nanoparticles” or “nanoscale particles” refers to very small particles compared with the spray-dried powder. This property enables the spray-dried powders to be coated with nanoscale particles. If, for example, spray-dried particles are produced having a mean diameter of about 5 μm, the nanoparticle should have a mean particle size of 1 nm-500 nm, or 5 nm-250 nm, or 10 nm-100 nm.

“Aerosil®” denotes nanoparticles of silicon dioxide (SiO₂) or of modified hydrophobic (acyl chain) silicon dioxide such as Aerosil® R812 made by Degussa. Other examples of nanoparticles are e.g. titanium dioxide (TiO₂).

By the term “biodegradable nanoparticles” are meant hereinafter nanoparticles that can be broken down in the human or animal body, preferably without producing harmful or unnatural breakdown products.

The term “dosage” or “dosages” refers to the amount of a substance, particularly a therapeutic active substance, that is delivered when an administration device such as an inhaler is used. The crucial factor for the dose is the proportion of substance, particularly active substance, in the powder and the amount of powder delivered. The delivered dose in the case of spray-dried powders can thus be adjusted either by changing the proportion of active substance in the spray-dried powder or by adding or mixing in an inert powder, i.e. one which is free from active substance.

The term “dilution” refers to a reduced dosage of a spray-dried powder, particularly a spray-dried powder containing an active substance.

The term “mixing component” means the substance that is fed into the spray dryer as a powder, apart from the spray-dried powder. The mixing component may consist of nanoscale or microscale particles or carriers.

Compositions According to the Invention

The present invention relates to a method of mixing spray-dried powders with nanoparticles, microscale particles and/or with carriers, characterised in that the mixing process is carried out in the spray dryer.

The present method is particularly characterised in that there is no transfer of the powder into a mixing apparatus after the spray-drying.

The present invention further relates to a method of mixing powders which is characterised by the following steps:
a. spraying/atomising a spray solution containing one or more substances that are to be sprayed and optionally one or more excipients into a compartment,
b. drying the resulting drops in the same compartment as in step (a),
c. introducing one or more other powders containing, e.g. carriers or nanoparticles into the same compartment as in step (b) under conditions in which s mixture is formed, and
d. collecting the particles formed.

A mixture is produced particularly if in the course of steps c or d both the spray-dried particles and the other powder components are combined in disaggregated form. This is preferably carried out during pneumatic atomising of the carriers or nanoparticles added in step c. A preferred mixing pressure for this pneumatic atomisation is 1.75 bar for a 1 mm or 2 mm slot width of the dispersing unit.

As an alternative to pneumatic atomisation through a mixing slot, mixing may be carried out in particular using other gas flow nozzles, such as a venturi nozzle, for example.

As an alternative to pneumatic disaggregation of the added carriers and nanoparticles, ultrasonically- or electrostatically-induced disaggregation may also be carried out.

In one particular embodiment the process according to the invention is characterised in that
a. the spray-dried powders are powders with a mean aerodynamic particle size (MMAD) of between 0.5-10 μm, 1-10 μm, preferably 2-7.5 μm,
b. the nanoparticles are particles with a mean particle size (MMD) of less than 500 nm, less than 200 nm or with an MMD between 1 nm-500 nm, 5-250 nm, preferably 10-100 nm and
c. the microscale particles are particles with a median aerodynamic particle size (MMAD) of between 0.5-10 μm, 1-10 μm, preferably 2-7.5 μm, and
d. the carriers are substances with a mean particle size (MMD) of more than 50 μm or between 50-200 μm, preferably 60-100 μm.

In a preferred embodiment, the process according to the invention is characterised in that the carriers have a proportion of at least 30% (w/w) of particles with a particle size of less than 100 μm.

In a particular embodiment, the process according to the invention is characterised in that at least 30% (w/w) of the carriers are smaller than 100 μm.

In another embodiment the method is characterised in that the nanoparticles or carriers are sugars, polyols or amino acids.

In a preferred embodiment the process is characterised in that the carrier is a pharmaceutically acceptable, crystalline excipient, such as for example a crystalline sugar, for example lactose monohydrate, glucose or chitosan or a crystalline polyol (for example mannitol).

In another embodiment the method is characterised in that the nanoparticles are inorganic compounds such as modified or unmodified silicon dioxide (SiO₂), titanium dioxide (TiO₂) or calcium carbonate (CaCO₃).

In a particularly preferred embodiment the method is characterised in that the nanoparticles are biodegradable nanoparticles. These include for example nanoscale monosaccharides or nanoscale polyols, such as glucose or mannitol, nanoscale di-, oligo- or polysaccharides, such as for example lactose monohydrate, saccharose, starch, amylose, amylopectin, hydrolysed starch, hydroxyethylstarch, carrageen, chitosan, or dextrans, nanoscale amino acids such as for example valine or glycine, nanoscale polymers such as for example gelatine, polyacrylates, polymethacrylates, polycyanoacrylates (e.g. Poly(isohexylcyanoacrylate), poly(methylcyanoacrylate), poly(ethylcyanoacrylate)), PLGA (poly(lactic-co-glycolic acid), polylactides, polyglycolides, polycaprolactones or human serum albumin (HSA) or nanoscale lipids, such as for example tripalmitin-containing or phosphatidylcholine-containing nanoparticles.

In another special embodiment the method is characterised in that the nanoparticles are not Aerosil® (silicon dioxide) and/or titanium dioxide.

In another embodiment the method according to the invention is characterised in that the compartment in step (a), (b) and (c) is a spray-dryer.

In another embodiment the method is characterised in that the drying in step (b) is carried out in a drying tower.

In a special embodiment the method is characterised in that step (b) is carried out by the cocurrent method.

In a special embodiment the method is characterised in that step (b) is not carried out by the countercurrent method.

In another embodiment the method is characterised in that the particles in step (d) are collected in a cyclone.

In another embodiment the method according to the invention is characterised in that the spray solution of step (a) is either an aqueous solution or a solution consisting of any desired pharmaceutically acceptable organic solvent.

In another embodiment the method according to the invention is characterised in that the drying medium in step (b) is either air or nitrogen.

In another embodiment the method according to the invention is characterised in that in the drying in step (b) the entry temperature of the drying gas is between 50° C. and 200° C. and the exit temperature of the drying gas after the drying process is between 25° C. and 150° C.

In another embodiment the process according to the invention is characterised in that the temperature load on the spray-dried powder is reduced by blowing in cool air after the drying (e.g. at the exit from the drying tower).

In another embodiment the method according to the invention is characterised in that one or more carriers or nanoparticles are introduced directly into a compartment through separate dispersing and metering units.

In another embodiment the method according to the invention is characterised in that one or more carriers or nanoparticles are pre-mixed and then fed directly into the spray dryer together through a dispersing and metering unit.

In another embodiment the process according to the invention is characterised in that no additional energy input is needed to disperse the spray-dried powder.

In another embodiment the method according to the invention is characterised in that two proteins are mixed together.

In a special embodiment the methods according to the invention are characterised in that methods of preparing detergents or inorganic mixtures are excluded, or methods in which inorganic powders are prepared by drying and then mixed with other powders are excluded.

In a special embodiment the methods according to the invention are characterised in that no detergents or inorganic mixtures are prepared, and no mixtures are prepared, in which inorganic powders are produced by drying and then mixed with another powder.

The present invention further relates to a method of coating spray-dried particles with nanoparticles, characterised in that one of the methods described according to the invention is used.

The present invention also relates to a method of preparing compositions or dosages containing a defined amount in percent by weight (w/w) of a spray-dried powder in the powder mixture, by admixing a carrier, characterised in that one of the methods described according to the invention is used.

In a preferred embodiment the methods according to the invention are characterised in that the spray-dried powder is a protein-containing powder.

In a preferred embodiment the protein is an antibody.

The invention further relates to a composition that has been prepared by one of the methods according to the invention.

In a special embodiment the composition is a composition for use as a medicament.

In a preferred embodiment the composition is used as an inhaled medicament.

In another embodiment the composition according to the invention is used to prepare a medicament for treating a pulmonary disease or a systemic disease.

The invention further relates to powder mixtures, characterised in that they have a spray-dried protein content of more than 1% (w/w), particularly more than 30% (w/w), 35% (w/w), 40% (w/w), 45% (w/w), 50% (w/w), 55% (w/w), 60% (w/w), 65% (w/w), more than 70% (w/w), more than 80% (w/w) and more than 90% (w/w) and comprise at least one nanoparticle or a carrier, the powder mixture having a fine particle fraction of more than 15% (w/w), more than 25% (w/w), more than 35% (w/w), more than 45% (w/w), more than 55% (w/w), more than 65% (w/w).

In a special embodiment the powder mixture according to the invention is characterised in that the protein content comprises antibodies.

### Examples

### Example 1

In this Example a spray solution was prepared, containing 70% IgG2 and 30% trehalose dihydrate, based on the solids content. The solids content of the solution was 3%. The spray solution was dried with a Büchi B-191 using a so-called High Performance Cyclone (HPC). Compared with the standard cyclone, the HPC has a lower precipitation threshold and hence a better precipitation efficiency, on account of its smaller diameter.

The drying conditions were:

entry temperature: 160° C.

spray rate of solution: 3.0 mL/min

atomiser gas rate: 700 L/h

The preparation of the mixtures was carried out directly in the drying tower by blowing in lactose monohydrate (Granulac 140) (see FIG. 1A). The dispersing of the lactose was carried out by a shear action at a slot (slot width 1 mm) The dispersing pressure was 1.75 bar.

3 different powders or powder mixtures were prepared.

TABLE 1

| | powder 1 | powder 2 | powder 3 |
|---|---|---|---|
| amount of spray-dried powder | 100 | 70 | 10 |
| in the mixture (% w/w) | | | |
| amount of Granulac 140 in the | 0 | 30 | 90 |
| mixture (% w/w) | | | |
| delivered mass, % | 69.5 | 79.6 | 98.5 |
| fine particle fraction, % | 12.3 | 24.2 | 28.7 |

Both in powder 2 and in powder 3, the aerodynamic properties (FPF and delivered mass) of the mixtures could be improved compared with the spray-dried powder without Granulac 140 (powder 1) (see FIG. 2). The fine particle fraction was determined by wet chemistry on the basis of the active substance in the capsule before delivery. The delivered mass is obtained from the difference in mass before and after expulsion of the powder from the powder inhaler (HandiHaler®).

FIG. 3 shows a scanning electron microscope image of the powder mixture of powder 2.

### Example 2

In this Example the homogeneity of the delivered dose of a mixture of spray-dried powder and a carrier (Granulac 140) was determined The parameters for spray drying were set analogously to those described for Example 1.

Composition of the powders:

TABLE 2

| | ST60 | ST63 |
|---|---|---|
| spray-dried powder | 70% (w/v) IgG2/ | 70% (w/v) IgG2/ |
| | 30% (w/v) trehalose | 30% (w/v) trehalose |
| carrier | Granulac 140 | — |
| mass ratio of carrier to | 9/1 | — |
| spray-dried powder | | |

TABLE 3

| | dose in percent | | | |
|---|---|---|---|---|
| | based on the | dose in percent | | |
| | weight of active | based on the | | |
| | substance placed | amount of protein | difference | delivered mass |
| powder ST60 | in the capsule | delivered | [% absolute] | in percent |
| measurement 1 | 87.0 | 86.3 | −0.7 | 97.7 |
| measurement 2 | 89.9 | 89.7 | −0.2 | 97.1 |
| measurement 3 | 92.8 | 91.8 | −1.0 | 98.0 |
| measurement 4 | 112.4 | 112.1 | −0.2 | 97.1 |
| measurement 5 | 119.2 | 120.0 | 0.8 | 96.3 |
| measurement 6 | 104.0 | 104.9 | 1.0 | 96.0 |
| measurement 7 | 86.3 | 86.5 | 0.1 | 96.8 |
| measurement 8 | 93.2 | 93.3 | 0.1 | 96.8 |
| measurement 9 | 111.5 | 112.9 | 1.5 | 95.7 |
| measurement 10 | 103.8 | 102.5 | −1.3 | 98.2 |
| min | 86.3 | 86.3 | −1.3 | 95.7 |
| max | 119.2 | 120.0 | 1.5 | 98.2 |
| rel. standard | 11.7 | 12.2 | | 0.9 |
| deviation | | | | |

TABLE 4

| | dose in percent | | | |
|---|---|---|---|---|
| | based on the | dose in percent | | |
| | weight of active | based on the | | |
| | substance placed | amount of | difference | delivered mass in |
| powder ST63 | in the capsule | protein delivered | [% absolute] | percent |
| measurement 1 | 90.0 | 109.1 | 19.1 | 74.4 |
| measurement 2 | 94.2 | 107.4 | 13.3 | 79.1 |
| measurement 3 | 106.5 | 107.3 | 0.8 | 89.5 |
| measurement 4 | 104.2 | 95.3 | −9.0 | 98.7 |
| measurement 5 | 96.2 | 101.4 | 5.2 | 85.6 |
| measurement 6 | 116.6 | 115.2 | −1.4 | 91.3 |
| measurement 7 | 78.3 | 77.9 | −0.4 | 90.6 |
| measurement 8 | 129.7 | 97.8 | −31.9 | 119.6 |
| measurement 9 | 112.3 | 113.1 | 0.8 | 89.5 |
| measurement 10 | 72.0 | 75.5 | 3.4 | 86.1 |
| min | 72.0 | 75.5 | −31.9 | 74.4 |
| max | 129.7 | 115.2 | 19.1 | 119.6 |
| rel. standard | 17.5 | 13.8 | | 13.6 |
| deviation | | | | |

By preparing the mixture from 90% Granulac 140 and 10% spray-dried powder it was possible to improve the homogeneity or uniformity of dosage compared with the spray-dried powder. As can be seen from Tables 3 and 4, both the delivered doses based on the weight of active substance placed in the capsule and on the delivered amount of protein are more homogeneous. Moreover, the delivery of the powder from the capsule is significantly improved by the preparation of the mixture.

### Example 3

In this Example the reproducibility of preparation of powder mixtures in the spray dryer was examined For this purpose, three batches of a powder formulation were prepared as described in Example 1. The Granulac 140 was fed in at a dispersing pressure of 1.75 bar and a slot width of 2 mm. Table 5 shows the fine particle fractions based on the amount weighed out as well as the delivered masses of powder from the inhaler. Both measuring parameters exhibit a very narrow range of fluctuations. This means that the mixing process in the spray dryer can be carried out in a very precise manner.

TABLE 5

| | Preparation number | | |
|---|---|---|---|
| | ST60 | ST61 | ST62 |
| spray-dried powder | 70% (w/v) IgG2/30% (w/v) | | |
| | trehalose | | |
| carrier | Granulac 140 | | |
| mass ratio of carrier/spray- | 9/1 | | |
| dried powder | — | | |
| FPF [%] | 28.7 | 24.4 | 24.6 |
| delivered mass [%] | 98.5 | 97.0 | 98.4 |

### Example 4

This Example shows various mixtures consisting of spray-dried powder and Aerosil R812.

FIG. 4 shows an example of a spray-dried powder coated with Aerosil.

FIG. 5 shows the fine particle fractions obtained for the various mixtures, depending on the dispersing pressure.

By coating with Aerosil R812 it is possible to increase the fine particle fraction.

### Example 5

This Example shows a mixture of spray-dried powder with hydrophobised silicon dioxide (Aerosil R 972, Degussa) and a mixture of highly dispersed hydrophilic silicon dioxide (Merck, Darmstadt, CAS no. 7631-86-9). The process conditions of spray-drying are shown in Table 6. The hydrophobic or hydrophilic nanoscale mixing component was supplied pneumatically. To do this, a storage vessel (total volume 1.1 L) was filled with 200 mL of nanoscale powder. Above the powder bed, 0.5 L/min of air was introduced tangentially into the storage vessel. To homogenise the powder bed, the powder was additionally stirred mechanically (300 rpm). The powder was then fed into the spray dryer through a venturi nozzle. The preliminary pressure of the venturi nozzle was 2 bar. Another vessel was interposed between the storage vessel and the venturi nozzle. Coarser clumps of particles were deposited in this vessel. Optionally, there is the possibility of introducing additional mixing air and also additional particle aerosols into the spray dryer through this vessel.

TABLE 6

| | |
|---|---|
| Spray dryer | Büchi B 191 |
| Entry temperature | 150° C. |
| Drying gas rate | 100% |
| Spray rate | 5 mL/min |
| Atomiser gas rate | 700 L/h |
| Cyclone | Standard |

In order to prepare the spray solution, 0.9 g of trehalose dihydrate were dissolved in about 70 ml of water. After dissolving, 15.4 ml of solution containing IgG1 (protein concentration: 104 mg/mL) were added and topped up to 100 ml with water. The average protein content in the powder after spray drying without the addition of a mixing component was 58%. After mixing with hydrophobic SiO₂, the proportion of protein in the powder was reduced to 47%. In the case of hydrophilic SiO₂, the protein content was 54%. Table 7 shows the aerodynamic properties of the different powders produced. The measurements were carried out as described in FIG. 2. In contrast to the method described therein, the fine particle fraction was determined gravimetrically by weighing the inserted capsule before and after the delivery of the powder into the measuring device.

TABLE 7

| | Without the addition | Hydrophobic | Hydrophilic SiO₂/ |
|---|---|---|---|
| | of a mixing | SiO₂/ | production |
| Mixing | component/ | production | batches: |
| component | production batch: N45 | batch: N34 | N40 and N43 |
| FPF, % | 26 | 59 | 50 |
| EM, % | 80 | 94 | 92 |
| MMAD, μm | 2.7 | 3.4 | 4.3 |

The increase in the gravimetrically determined FPF by the addition of nanoscale particles, of 33% FPF in the case of hydrophobic SiO₂ and 29% FPF in the case of hydrophilic SiO₂, is to be put down essentially to an improvement in the surface quality of the spray dried powders, as the proportion of the additional component in the mixture is less than 11% and hence the increase in the FPF is not due primarily to an increased proportion by mass of inhalable SiO₂ in the powder.

This example shows the possibility of adjusting the protein content in the powder and hence the dose of active substance by directly mixing microscale excipients with the spray-dried powder.

The spray conditions for producing the spray-dried powder are described in Table 8.

In order to prepare the spray solution, 0.9g of trehalose dihydrate were dissolved in about 70 ml of water. After dissolving, 14.5 ml of solution containing IgG1 was added (protein concentration: 104 mg/mL) and topped up to 100 ml with water. The proportion of protein in the powder after spray drying without the addition of a mixing component was 60%.

TABLE 8

| | |
|---|---|
| Spray dryer | Büchi B 191 |
| Entry temperature | 150° C. |
| Drying gas rate | 100% |
| Spray rate | 5 mL/min |
| Atomiser gas rate | 700 L/h |
| Cyclone | Standard |

The mixing component used was lactose monohydrate which was micronised by grinding. After micronisation, the sugar had an MMAD of 3.9 μm and a gravimetrically determined fine particle fraction of 14%.

The micronised lactose monohydrate was metered using a metering screw (ZD9F, made by Three-Tec). The discharged powder was fed into a venturi nozzle with an air current of 20 L/min. The preliminary pressure of the venturi nozzle was 0.69 bar. As described in Table 9, two powder mixtures were prepared with different metering rates of the metering screw. The fine particle fraction or the amount of protein in the fine particle fraction was determined by wet chemistry. For this, three capsules were placed in the impactor inlet (type 3306/TSI) and then the filter downstream of the impactor nozzle was analysed. The method is described in FIG. 2. A buffer consisting of 25 mM histidine/1.6 mM glycine, pH 6.0, was used as the reconstitution medium.

The mixing ratio between the spray dried powder and the micronised lactose monohydrate is shown in Table 9. In powder 2, a protein content in the powder of 38.8% is obtained, for example, by mixing the two components in the proportions 62% (w/w) of spray dried powder and 38% (w/w) of micronised lactose monohydrate.

As shown in Table 9, the protein content in the fine particle fraction could be reduced significantly by mixing with the mixing component.

The protein contents in the powder and hence the mixing ratio after delivery using the Handihaler in the fine particle fraction is almost identical to the starting mixture. Table 10 shows the protein content of the starting mixture and in the fine particle fraction determined The protein content in the fine particle fraction is obtained from the protein content in the FPF determined by wet chemistry, based on the quantity of powder in the FPF. This shows that in particular microscale excipients such as micronised lactose monohydrate, for example, are well suited to preparing powder mixtures in the spray dryer as the powder components behave similarly in aerodynamic terms and hence unmixing processes are suppressed.

TABLE 9

| | Powder 1 | Powder 2 | Powder 3 |
|---|---|---|---|
| Metering rate CD9F, rpm | No ingredients | 5 | 8 |
| | mixed in | | |
| Protein content in the | 60.2 | 38.8 | 28.5 |
| powder after manufacture, % | | | |
| FPF, % | 22 | 17 | 14 |
| Amount of protein in the | 5.5 | 3.9 | 2.2 |
| fine particle fraction, mg | | | |
| Mixing ratio of spray dried | — | 62/38 | 47/53 |
| powder to mixing | | | |
| component % w/w | | | |

TABLE 10

| | Protein content of the | Protein content of the |
|---|---|---|
| | powder mixture after | powder mixture in the fine |
| | spray drying, % | particle fraction, % |
| Powder 2 | 37.8 | 37.9 |
| Powder 3 | 28.5 | 29.5 |

A spray solution was prepared corresponding to the weights as described in Table 11. The spray conditions for preparing the spray dried powder are described in Table 12.

TABLE 11

| | Weight in grams for 1 litre of |
|---|---|
| | purified water |
| Trehalose dihydrate | 84 |
| L-histidine | 0.68 |
| L-histidine HCL monohydrate | 3.27 |
| Polysorbate 80 | 0.2 |
| EDTA | 0.1 |
| Bovine Serum Albumin | 0.82 |

TABLE 12

| | |
|---|---|
| Spray dryer | Büchi B 191 |
| Entry temperature | 150° C. |
| Drying gas rate | 100% |
| Spray rate | 5 mL/min |
| Atomiser gas rate | 700 L/h |
| Cyclone | Standard |

The spray dried powder was mixed with various Pharmatoses (DMV) in the spray dryer (see Table 13). The ingredients were metered in using a metering screw (ZD9F, Three-Tec) at 10 rpm. The powder discharged was introduced directly into the spray dryer with an air current of 20 L/min.

As can be seen from Table 13, the mixing ratio obtained is critically dependent on the particle size of the mixing component used. Specially coarser carriers such as Pharmatose 50M and Pharmatose 90M are less suitable for in-line mixing as there is a strong tendency for unmixing processes to occur in these. The mixing component used should have at most the particle size corresponding to Pharmatose 125M. Smaller particles are preferable particularly when larger amounts of mixing component are added.

TABLE 13

| | Powder 1 | Powder 2 | Powder 3 | Powder 4 |
|---|---|---|---|---|
| Mixing | No | Pharmatose | Pharmatose | Pharmatose |
| component | ingredients | 50M | 90M | 125M |
| | mixed in | | | |
| Proportion of | — | 65 | 63 | 62 |
| mixing | | | | |
| component in | | | | |
| the powder, | | | | |
| % (w/w) | | | | |
| Theoretical | — | 0.37 | 0.40 | 0.41 |
| protein charge | | | | |
| Protein charge | 1.07 | 0.08 | 0.12 | 0.26 |
| measured | | | | |
| in the powder, | | | | |
| % (w/w) | | | | |
| Recovery, % | — | 21 | 30 | 64 |

1. A method of mixing spray-dried powder with nanoparticles, microscale particles and/or with carriers, the comprising mixing the spray-dried powder with nanoparticles, microscale particles and/or with carriers in a spray dryer.

2. The method according to claim 1, wherein there is no transfer of the powder into a mixing apparatus after spray-drying.

3. A method of mixing powders, comprising the following steps:
a. spraying/atomising a spray solution containing one or more substances that are to be sprayed, as well as optionally one or more excipients, into a compartment,
b. drying the resulting drops in the same compartment as in step (a),
c. introducing one or more other powders containing e.g. carriers or nanoparticles into the same compartment as in step (b) under conditions in which a mixture is formed and
d. collecting the particles formed.

4. The method according to claim 3, wherein the
a. the spray-dried powders are powders with a mean aerodynamic particle size (MMAD) of between 0.5- 10 μm,
b. the nanoparticles are particles with a mean particle size (MMD) of less than 500 nm, and
c. the microscale particles are particles with a median aerodynamic particle size (MMAD) of between 0.5-10 μm, and
d. the carriers are substances with a mean particle size (MMD) of more than 50 μm.

5. The method according to claim 4, wherein the carriers have a proportion of at least 30% (w/w) of particles with a particle size of less than 100 μm.

6. The method according to claim 5, wherein the nanoparticles or carriers are sugars, polyols or amino acids.

7. The method according to claim 6, wherein the carrier is a pharmaceutically acceptable crystalline excipient.

8. The method according to claim 7, wherein the excipient is a crystalline sugar such as lactose monohydrate, glucose, chitosan or a crystalline polyol.

9. The method according to claim 6, wherein the nanoparticles are silicon dioxide (SiO₂), titanium oxide (TiO₂) or calcium carbonate (CaCO₃) in modified or unmodified form.

10. The method according to claim 6, wherein the nanoparticles are biodegradable nanoparticles.

11. The method according to claim 10, wherein the biodegradable nanoparticles are nanoscale monosaccharides, nanoscale polyols, nanoscale di-, oligo- or polysaccharides, nanoscale amino acids, nanoscale polymers or nanoscale lipids.

12. The method according to claim 11, wherein the compartment of step (a), (b) and (c) is a spray dryer.

13. The method according to claim 12, wherein the drying in step (b) is carried out in a drying tower.

14. The method according to claim 13, wherein step (b) is carried out by the cocurrent method.

15. The method according to claim 14, wherein the particles in step (d) are collected in a cyclone.

16. The method according to claim 15, wherein the spray solution from step (a) is either an aqueous solution or a solution consisting of any desired pharmaceutically acceptable organic solvent.

17. The method according to claim 16, wherein the drying medium in step (b) is either air or nitrogen.

18. The method according to claim 17, wherein during the drying in step (b), the entry temperature of the drying gas is between 50° C. and 200° C. and the exit temperature of the drying gas after the drying process is between 25° C. and 150° C.

19. The method according to claim 18, wherein the temperature loading of the spray-dried powder is reduced by blowing in cool air after the drying (e.g. at the exit from the drying tower).

20. The method according to claim 19, wherein one or more carriers or nanoparticles are introduced directly into a compartment through separate dispersing and metering units.

21. The method according to claim 20, wherein one or more carriers or nanoparticles are pre-mixed and then introduced together into the spray dryer directly through a dispersing and metering unit.

22. A method of coating spray-dried particles with nanoparticles, wherein the method according to claim 21 is used.

23. The method according to claim 21 further comprising preparing compositions or dosages, containing a defined amount (in w/w) of spray-dried powder, by admixing a carrier.

24. The method according to claim 23, wherein the spray-dried powder is a protein-containing powder.

25. The method according to claim 24, wherein the protein is an antibody.

26. (canceled)

27. A pharmaceutically acceptable composition produced by the process according to claim 25.

28. The pharmaceutically acceptable composition according to claim 27 for use as an inhaled medicament.

29-31. (canceled)

32. A method of treating of a pulmonary disease or a systemic disease comprising administering a composition according to claim 27.

33. A powder mixture, comprising a spray-dried protein content chosen from
more than 1% (w/w),
more than 30% (w/w),
more than 35% (w/w),
more than 40% (w/w),
more than 45% (w/w),
more than 50% (w/w),
more than 55% (w/w),
more than 60% (w/w),
more than 65% (w/w),
more than 70% (w/w),
more than 80% (w/w) and
more than 90% (w/w),
and further comprising at least one nanoparticle or a carrier, the powder mixture having a fine particle fraction chosen from
more than 15% (w/w), more than 25% (w/w), more than 35% (w/w), more than 45% (w/w), more than 55% (w/w) and more than 65% (w/w).

34. The powder mixture according to claim 33, wherein the protein content comprises antibodies.

35. The method according to claim 4, wherein the
a. the spray-dried powders are powders with a mean aerodynamic particle size (MMAD) of between 1-10 μm,
b. the nanoparticles are particles with a mean particle size (MMD)-less than 200 nm and
c. the microscale particles are particles with a median aerodynamic particle size (MMAD) of between 1-10 μm and
d. the carriers are substances with a mean particle size (MMD) of between 50-200 μm.

36. The method according to claim 35 wherein the
a. the spray-dried powders are powders with a mean aerodynamic particle size (MMAD) of between 2-7.5 μm,
b. the nanoparticles are particles with a mean particle size (MMD) of between 1 nm -500 nm and
c. the microscale particles are particles with a median aerodynamic particle size (MMAD) of between 2-7.5 μm, and
d. the carriers are substances with a mean particle size (MMD) of between 60-100 μm.

37. The method according to claim 36 wherein the
b. the nanoparticles are particles with a mean particle size (MMD) of between 5-250 nm.

38. The method according to claim 37 wherein the
b. the nanoparticles are particles with a mean particle size (MMD) of between 10-100 nm.

39. The method according to claim 11, wherein the biodegradable nanoparticles are glucose, mannitol, lactose monohydrate, saccharose, starch, amylose, amylopectin, hydrolysed starch, hydroxyethylstarch, carrageen, chitosan, dextrans, valine, glycine, gelatine, polyacrylates, polymethacrylates, poly(isohexylcyanoacrylate), poly(methylcyanoacrylate), poly(ethylcyanoacrylate)), PLGA (poly(lactic-co-glycolic acid), polylactides, polyglycolides, polycaprolactones, human serum albumin (HSA), tripalmitin-containing or phosphatidylcholine-containing nanoparticles.
